(19) United States
(12) Patent Application Publication
(10) Pub. No.: US 2006/0035252 A1
(43) Pub. Date: Feb. 16, 2006

(54) Methods and workflows for selecting genetic markers utilizing software tool

(75) Inventors: Francisco M. De La Vega, San Mateo, CA (US); Hadar Isaac, Los Altos, CA (US)

Correspondence Address: HARNESS, DICKEY & PIERCE, P.L.C., P.O. BOX 828, BLOOMFIELD HILLS, MI, 48303, US

(73) Assignee: APPLERA CORPORATION, Foster City, CA (US)

(21) Appl. No.: 11/181,591

(22) Filed: Jul. 14, 2005

Related U.S. Application Data
(63) Continuation-in-part of application No. 10/833,000, filed on Apr. 28, 2004.
(60) Provisional application No. 60/466,310, filed on Apr. 28, 2003.
(60) Provisional application No. 60/588,274, filed on Jul. 14, 2004.
(60) Provisional application No. 60/619,145, filed on Oct. 15, 2004.

(51) Int. Cl. C12Q 1/68; G06F 19/00; G01N 33/48; G01N 33/50

(52) U.S. Cl. 435/6; 702/20

(57) ABSTRACT

A visual tool facilitates selecting SNPs for genotyping experiments comprises a first memory containing a datastore of pre-calculated linkage disequilibrium map information; a second memory containing a datastore of haplotype block information; and a third memory containing at least one set of tagging SNPs. A graphical user interface provides visualization of SNPs, integrated with a physical genome map. A stepwise selection tool associated with the graphical user interface facilitates selection of tagging SNPs by selectively using the information in at least one of the first, second and third memories.

### CROSS REFERENCE TO RELATED APPLICATIONS

This application is a continuation-in-part of U.S. patent application Ser. No. 10/833,000, entitled “Methodology and Graphical User Interface to Visualize Genomic Information,” filed Apr. 28, 2004, which claimed benefit of U.S. Provisional patent application Ser. No. 60/466,310.

This application claims the benefit of U.S. Provisional Patent application Ser. No. 60/588,274, entitled “Tagging SNP Methods and LD-Guided Selection of Markers for Association Studies, filed Jul. 14, 2004. This application further claims the benefit of U.S. Provisional Patent application Ser. No. 60/619,145, entitled “Methods and Workflows for Selecting Genetic Markers,” filed Oct. 15, 2004.

The disclosures of all aforesaid related applications and provisional applications are hereby incorporated by reference.

### Introduction

SNPs are useful markers for genetic association studies that strive, by means of the statistical association of neighboring alleles or linkage disequilibrium (LD), to localize the genes involved in disease susceptibility or adverse reactions to drugs. Although SNPs are abundant in the human genome, and large databases of candidate SNPs are available for selecting markers across the genome, not all candidate polymorphisms are suitable for selection as markers in genetic studies and for the development of genotyping assays. It has been reported several times in the literature that typically only 50% of SNPs selected at random from dbSNP yield working assays, which results in significant delays and expense.

There are several reasons for this high failure rate: (1) Many of the SNP records in public databases are candidate variants discovered with low-quality data (e.g., single EST reads), which often prove to be sequence or assembly artifacts or rare mutations; (2) Many SNPs are harbored in repeats or duplicated regions in the genome; assays directed to those regions result either in no signal, or they report all samples as heterozygous; (3) Even if the SNP proves to be a true variant, some variants provide no information (i.e. not heterozygous) about a given population.

The increased availability of validated SNPs whose allele frequency has been previously determined in reference samples from major populations helps alleviate some of these problems. More than two years ago, Applied Biosystems set out to validate more than 250,000 gene-centric SNPs, with the goal of creating a resource for candidate-gene, and candidate-region, genetic-association studies. The result was the release of TaqMan® Assays-on-Demand™ SNP Genotyping Products (now known as TaqMan® Validated SNP Genotyping Assays), comprising more than 150,000 assays with allele frequency information determined from African-American, Caucasian, Chinese, and Japanese individuals. These validated, ready-to-use-assays help ensure that studies using markers selected for genes or regions of interest will be successful.

More recently, the HapMap project has been funded to genotype more than one million SNPs distributed across the entire genome in four reference populations. Together these resources provide researchers with a large selection of validated SNPs for association mapping studies. For SNPs not included in the Applied Biosystems collection of validated assays, custom assays can be ordered through the Applied Biosystems Custom TaqMan® SNP Genotyping Service (previously TaqMan® Assay-by-Design® Service). Furthermore, researchers can select from a growing list of TaqMan Pre-Designed SNP Genotyping Assays, which have been computationally pre-screened for repeats and assembly artifacts, adjacent SNPs, and for the uniqueness of their amplicons (and in the case of Human SNPs, are functionally tested at manufacturing), to improve the probability of assay success. The latter set of assays is particularly useful for regions or genes not fully covered by the validated assay collection, or when higher density of markers is desirable.

An important factor to consider when selecting SNPs for genetic studies is how much information they provide for a given population. The most useful markers require relatively high heterozygosity in the study population, with a minor allele frequency of at least 5%. However, some areas of the genome may lack a sufficient number of validated SNPs for which the allele frequency in a reference sample has been established. In such cases, candidate SNPs can be prioritized based on evidence of independent discovery in two or more source s (the so-called “double hit” SNPs. For example, a SNP discovered by The SNP Consortium and reported to dbSNP while independently discovered by Celera Genomics during the sequencing of the Human genome, qualifies as a double hit SNP. By querying the Celera Human RefSNP database, the Celera Discovery System (CDS) can analyze the cross-references between two such discoveries. In addition to being confirmed as real variations, these double-hit SNPs are also likely to be highly heterozygous, as they typically have been ascertained in a small sample size (fewer than 5 individuals).

For genetic association studies, SNPs must be selected to maximize the probability that the unknown causative mutation is in significant LD with at least one of the markers genotyped in the study. Empirical studies have shown that LD can extend for tens of kilobases, suggesting that selecting evenly spaced SNPs with a density of, for example, one SNP per 10 kb, might be a reasonable means of choosing markers. That was precisely the approach selected for developing the 150,000 TaqMan Validated SNP Genotyping Assays. Analysis of the 40 million genotypes collected during the validation process, however, as well as reports by others, has shown that LD between SNPs varies tremendously across the genome, suggesting that a SNP selection process based exclusively on physical distance between the markers is not optimal.

As a result, another method of marker selection based on the observed empirical patterns of LD and analogous to the genetic recombination maps used for marker selection in linkage studies has been proposed. This method consists in a metric LD map that places SNPs in locations proportional to the extent of LD between adjacent markers and provides an intuitive means of spacing markers evenly across regions of interest. It also enables the detection of regions where, because of recombination, LD breaks down faster requiring additional markers. Furthermore, reports of blocks of high LD with limited haplotype diversity suggest that selecting a subset of SNPs with the ability to “tag” common haplotypes in a region (so-called “tagging” SNPs) could be a suitable strategy for selecting markers in these regions. A number of metrics to evaluate the correlation of the SNPs in a region of high LD aimed to select tagging SNPs have been suggested and an efficient, scalable algorithm framework to perform optimal selection of tagging SNPs with large datasets is now available.

The goal of selecting the correct SNP coverage is to provide the statistical power required to detect the association. When selecting SNPs for a study, integrating all the criteria described above can be challenging, even with the current availability of larger number of validated SNPs and empirical LD data. In particular, the algorithms required to analyze LD, develop LD maps, select haplotype-tagging SNPs, estimate power, and so on, are rather specialized. In addition, the necessary SNP annotations (e.g., allele frequency, double-hit status, suitability for a genotyping platform) are deposited in heterogeneous data sources.

Thus, from a practical standpoint, selecting the most suitable set of SNPs to allow genetic research to proceed in an efficient, cost-effective manner can be overwhelming. This is due, in part, to the millions of SNPs currently listed in public databases. Once a set of SNPs is selected, researchers have heretofore lacked a rapid way to obtain reliable, predictable assays for multiple SNPs that work together under the same experimental conditions.

### SUMMARY

To address these and other practical concerns in selecting SNPs for genotyping experiments, we have developed a set of methods and workflows for selecting genetic markers using a visual tool. In one embodiment, the visual tool to facilitate selecting SNPs for genotyping experiments comprises a first memory containing a datastore of pre-calculated linkage disequilibrium map information; a second memory containing a datastore of haplotype block information; and a third memory containing at least one set of tagging SNPs. A graphical user interface provides visualization of SNPs, integrated with a physical genome map. A stepwise selection tool associated with the graphical user interface facilitates selection of tagging SNPs by selectively using the information in at least one of the first, second and third memories. These and other features of the present teachings are set forth herein.

### BRIEF DESCRIPTION OF THE DRAWINGS

The skilled artisan will understand that the drawings, described below are for illustration purposes only. The drawings are not intended to limit the scope of the present teachings in any way.

FIG. 1 illustrates an exemplary SNPbrowser main visualization panel and graphical user interface;

FIG. 2 depicts an implementation of a step-by-step wizard deployed on SNPbrowser's Workflow selection panel;

FIG. 3 depicts a batch search for genomic locations by list of gene IDs;

FIG. 4 depicts an exemplary result of batch search using gene IDs, where the result list is “clickable’ on the SNPbrowser to immediately pan and zoom to the region of interest;

FIG. 5 illustrates a SNPbrowser visualization panel after zooming to region of interest. Validated SNPs are represented as blue horizontal sticks, whereas non-validated SNPs are represented as gray lines with gray IDs. An asterisk at the end of the non-validated SNP indicates “double-hit” status;

FIG. 6 illustrates the selection of a coordinate system and spacing criteria on the wizard;

FIG. 7 depicts a wizard panel to select options of the SNP prioritization scheme;

FIG. 8 is a flowchart of an algorithm for prioritization of validated SNPs;

FIG. 9 is a flowchart of an algorithm for prioritization of non-validated SNPs;

FIG. 10 illustrates a SNPbrowser visualization panel in which selected SNPs are indicated as highlighted red sticks. The red bar below close to the coordinate axis indicates the largest gap and if the spacing spec was fulfilled (in this case red indicates the largest gap is over the specification; alternatively green is presented);

FIG. 11 illustrates an exemplary graphical user interface useful to review the results of the selection and to iteratively change/explore the effect of some selection parameters change. Clicking “back” allows to quickly reselect options selected on earlier stages of the workflow wizard. The red square is a visual cue to indicate how well the algorithm was able to fulfill the spacing requirements (in this case red means the largest gap is over the specification);

FIG. 12 illustrates how a final list of selected markers appears in the “shopping basket” window. SNP ID's are sent to the shopping basket by clicking the “add” button from the final wizard screen. Clicking on the list spawns a highlighter showing the location of the marker (horizontal yellow line at left);

FIG. 13 illustrates correlation metric selection on the wizard;

FIG. 14 is a flowchart of an algorithm to eliminate SNPs by genotype correlation. Note that SNP comparison routine is detailed in FIG. 15;

FIG. 15 is a detailed flowchart of the SNP comparison routine illustrated to in FIG. 14;

FIG. 16 depicts selection of a correlation parameter via slider in an exemplary wizard screen for haplotype-based methods. Interactive tuning can also be performed from here with real time visualization feedback;

FIG. 17 depicts selection of correlation and MAF parameters via sliders in an exemplary wizard screen for genotype correlation method. Interactive tuning can also be performed from here with real time visualization feedback;

FIG. 18 shows the visualization of tagging relationships on SNPbrowser panel using yellow arcs;

FIG. 19 illustrates the genotype correlation wizard display, showing SNPs for which the samples show a genotypic correlation of 100%. In this instance 44 SNPs, of which 14 SNPs can be eliminated as their correlation with the selected tagging SNPs is 100%;

FIG. 20 depicts how the sliding window for each SNP analyzes only SNP sets within 1 LDU of the SNP that is being tagged;

FIG. 21 illustrates the pairwise r² method: Each SNP is assessed against the target SNP to determine the pairwise r²;

FIG. 22 is a spreadsheet illustrating the SNPs with various r² values, Green indicates SNPs with an r² greater than or equal to 0.95; Yellow indicates a minimal SNP set (i.e., 11 SNPs are reduced to a tagging set of four, which predict all SNPs with an r² greater than or equal to 0.95;

FIG. 23 illustrates how the haplotype R² method calculates the predictive ability of each putative haplotype; Blue indicates the SNP being tagged; Black indicates the SNPs used to calculate haplotype R²;

FIG. 24 is a user interface screen illustrating the prioritization schema for density selection. Note the tool tip for double-hit SNPs;

FIG. 25 illustrates the region encompassing the LIM gene chromosome 4(95,819,640-96,056,891 bp), which is based on Build NCBI b34;

FIG. 26 illustrates how tagging SNPs are selected using the SNP wizard and the pairwise r² method. Black bars represent selected SNPs; and

FIG. 27 is a software block diagram of one implementation of a visual tool to facilitate selecting SNPs for genotyping experiments.

### DESCRIPTION OF VARIOUS EMBODIMENTS

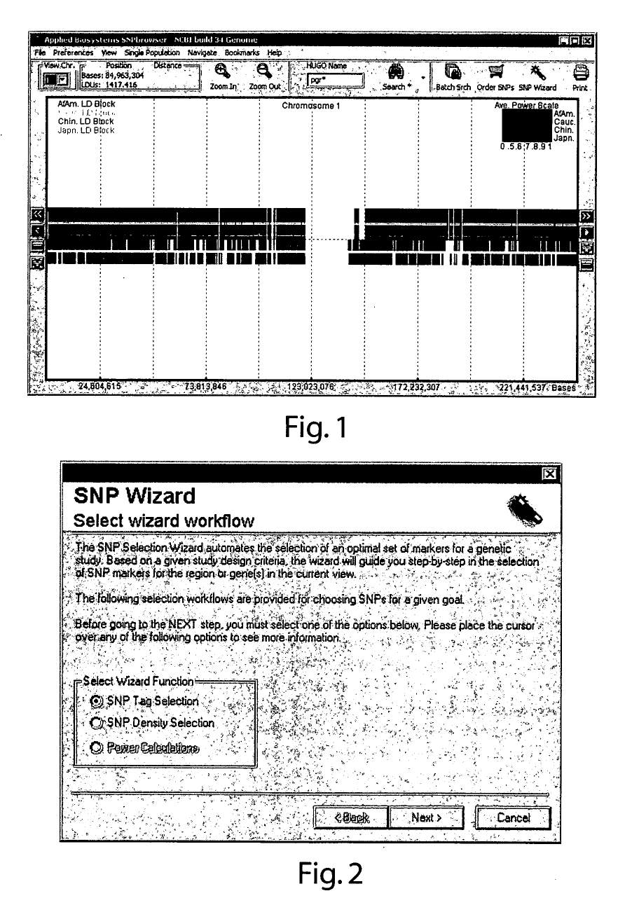

To simplify the complexity of selecting the appropriate SNP markers for genetic studies, we have developed a software tool we call the SNPbrowser. SNPbrowser is a tool to assist in the knowledge-based selection of markers for association studies. SNPbrowser may be implemented as a software tool that integrates all data and methodologies discussed above and that permits visualization of all relevant data points as well as the empirically observed LD. The basic visualization strategies utilized by SNPbrowser to present the locations of the SNPs, genes, LD maps, LD/haplotype blocks, the results of power calculations, as well as the basic features of the user interface and search and navigation facilities (FIG. 1), are further discussed in U.S. patent application Ser. No. 10/833,000, entitled “Methodology and Graphical User Interface to Visualize Genomic Information, which is hereby incorporated by reference.

In the present teachings, we further devise a number of SNP selection workflows that may be implemented as easy-to-use, step-by-step “wizards” within the a software tool, such as the SNPbrowser software. The tool allows researchers to prioritize their selection of validated, off-the-shelf TaqMan SNP Genotyping Assays, and supplement any gaps with pre-designed, functionally tested assays, to help ensure the highest probability of success of an association study. Additionally, the wizards can generate lists of SNPs, based on a number of genotyping approaches. Once a number of SNPs are selected, a few mouse clicks is all that is required to order assays products through an online store, such as the Applied Biosystems online store.

The selection methods described in the present teachings can be divided in the following basic workflows (cf. FIG. 2):
SNP selection by density or spacing
Selection of SNP “tags” or minimum informative subsets
Combinations thereof.
   These basic workflows can be used as building blocks to generate more complex workflows with diverse applications.

In these workflows, SNPs can be prioritized, taking into account the availability of validated, off-the-shelf assays, previous SNP validation data, double-hit status, and SNP type, in order to increase the probability of successfully utilizing the SNPs as markers in a genetic study.

I. SNP Density Selection Workflow

In the SNP density selection workflow (accessed via the SNP Density Selection button, shown in FIG. 2), markers are selected across a genomic region from a pool of validated and non-validated SNPs, with the goal of selecting the fewest number of evenly spaced markers (a “picket fence”). The coordinate system used to assess the spacing and distribution of SNPs can be either the physical map or the metric LD map described in the literature. See for example, Maniatis et al, “The first linkage disequilibrium (LD) maps: delineation of hot and cold blocks by diplotype analysis,” Proc. Natl, Acad. Sci. USA 99:2228-2233 (2002). The density selection workflow is useful to supplement Validated SNPs with additional SNPs when their density is not sufficient, or to select SNPs in a picket-fence fashion.

This workflow may be comprised of the following steps:
1) Selection of the genomic region(s) of interest
2) Selection of the coordinate system to place the markers
3) Selection of the target spacing that is desired
4) Selecting the filtering and prioritization scheme of the available candidate SNPs on the region
5) Selection of the fewest number of SNPs to meet the required spacing taking into account the prioritization scheme
6) Visualizing the result of the marker selection
7) Fine tuning of some of the selection parameters based on visual feedback and re-selection of markers
8) Create final list of selected SNP markers
9) Order assays for selected markers, e.g. linking to online store

Steps 6 to 7 are optional. A more detailed description of each step, alongside snapshots of a “wizard”-like software implementation follows.

Step 1: Selection of Genomic Region(s) of Interest.

The first step involves selecting the genomic region of interest. Typically this would be a contiguous chromosomal segment including one or more genes, but it could encompass an entire chromosome or genome. These regions are usually derived from a list of candidate genes for an association study, or can also be derived from candidate regions resulting from a previous linkage mapping study.

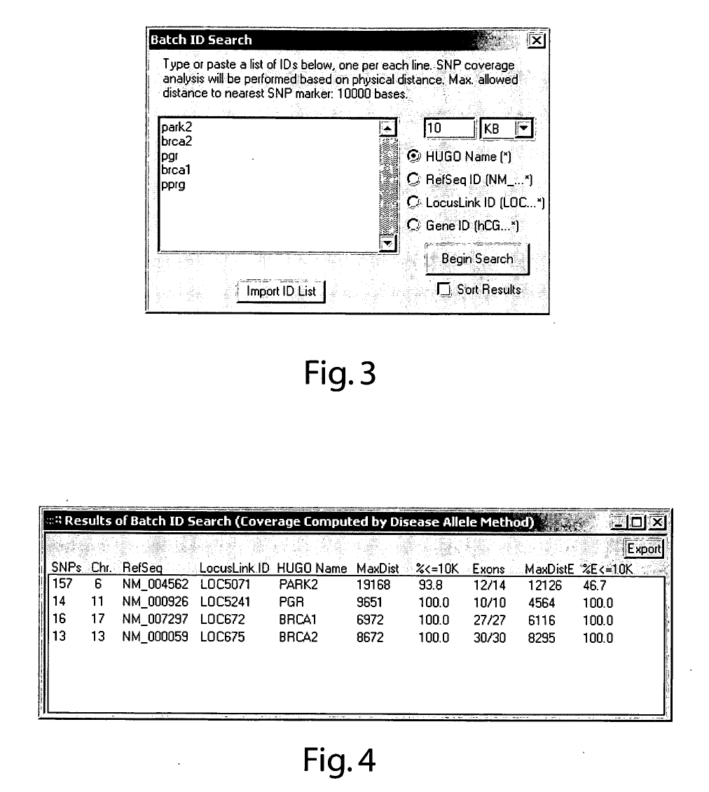

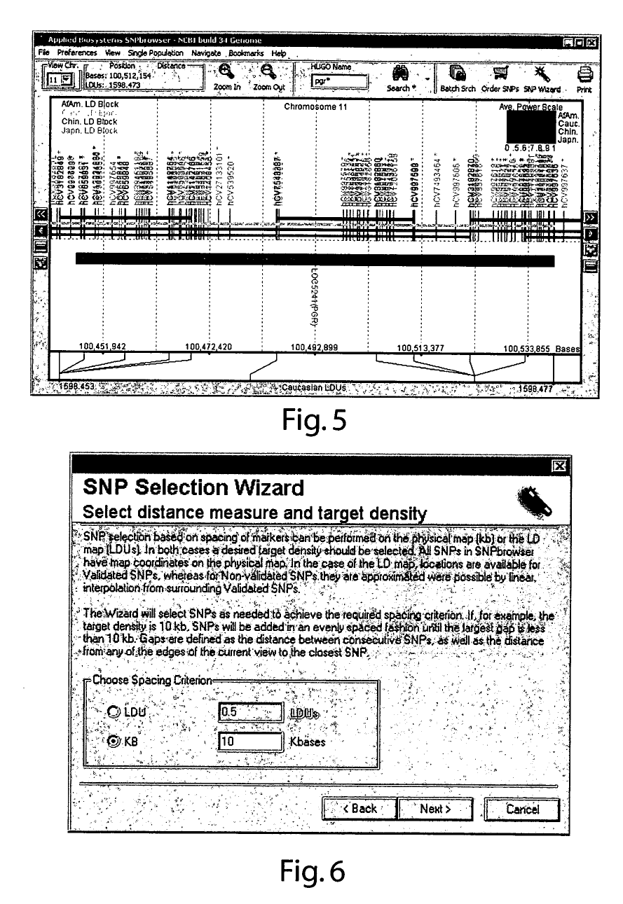

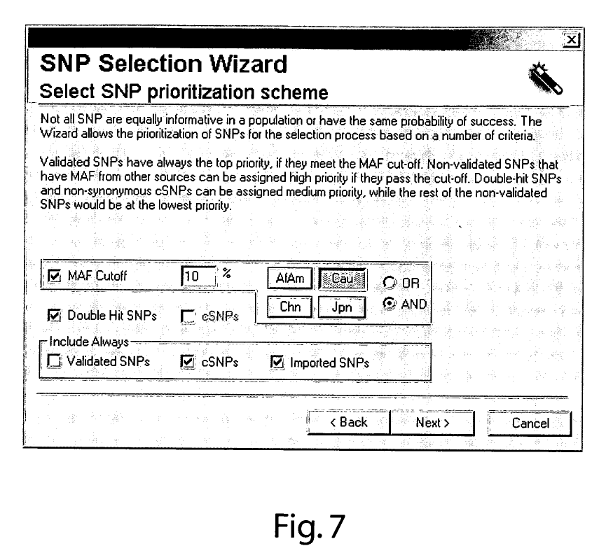

Typically, a search device will be used to zoom and pan into the region of interest. Searches can be performed on a per-region basis, or as a batch search from which one can navigate (pan and zoom) to each region of interest (FIGS. 3-5).

Step 2: Selection of the Coordinate System to Place the Markers

SNP selection based on spacing of markers can be performed on the physical map (kb) or on the metric LD map (LDUs) described in the Maniatis et al reference cited above (see FIG. 6). Other coordinates systems that are meaningful for the type of genetic study can be used as well, e.g. cM. All SNPs in SNPbrowser have map coordinates on the physical map. In the case of the LD map, locations are available for Validated SNPs, whereas for Non-validated SNPs they are approximated were possible by linear interpolation from surrounding Validated SNPs.

Step 3: Selection of the Target Spacing that is Desired

Once the coordinate system is selected a desired target density should be selected in the corresponding units (e.g. kb or LDUs). This number would represent the ideal spacing that the user wishes to attain, but also signifies the threshold above which spacing between two SNPs is not optimal (see FIG. 6). At this time a minimum spacing can also be specified, implying that in any case SNPs spaced less than this minimum should not be selected.

Step 4: Selecting the Filtering and Prioritization Scheme of the Available Candidate SNPs on the Region

As described above, not all SNPs are equally informative in a population or have the same probability of success. Prioritization of SNPs for the selection process can be achieved based on a number of criteria. Validated SNPs for which functionally tested assays are available typically have the top priority; optionally they may be required to meet a minor allele frequency (MAF) cut-off in the population of interest, or in a related population. Non-validated SNPs that have MAF information from other sources can be assigned high priority if they pass the cut-off even if a validated assay is not at hand. Double-hit SNPs (as described above) and non-synonymous coding SNPs can be assigned medium priority, while the rest of the non-validated SNPs typically would be at the lowest priority. All these criteria can be combined or used independently, and their priorities could be adjusted depending on one's objectives and the type of study.

Exemplary Prioritization Algorithm.

Each SNP, whether validated or non-validated may be assigned one out of six possible prioritization types before beginning the gap filling selection process:
“Free marker” This SNP will always be selected by the wizard. There will be no regard to its location or usefulness. The four other types below will later be considered for possible selection based on their usefulness in supplanting the “Free markers”.
High Priority Highest priority SNP of all those which are not a “Free marker”. This type will be considered as first choice in selection when SNPs are needed in addition to the “Free markers”.
Medium Priority Second highest priority SNP of all those which are not a “Free marker”. This type will be considered when SNPs are needed in addition to the “Free markers” and “High Priority” SNPs, in order to achieve the maximum gap requirement.
Low Priority Low priority SNP. This type will be considered when SNPs are needed in addition to the “Free markers”, “High Priority” SNPs, and “Medium Priority” SNPS, in order to achieve the maximum gap requirement.
No Priority This type will be considered when SNPs are needed in addition to the “Free markers” as well as the three levels of priority SNPs, in order to achieve the maximum gap requirement.
Discard This SNP will be ignored, never to be selected by the wizard, regardless of its position and usefulness in filling gaps.

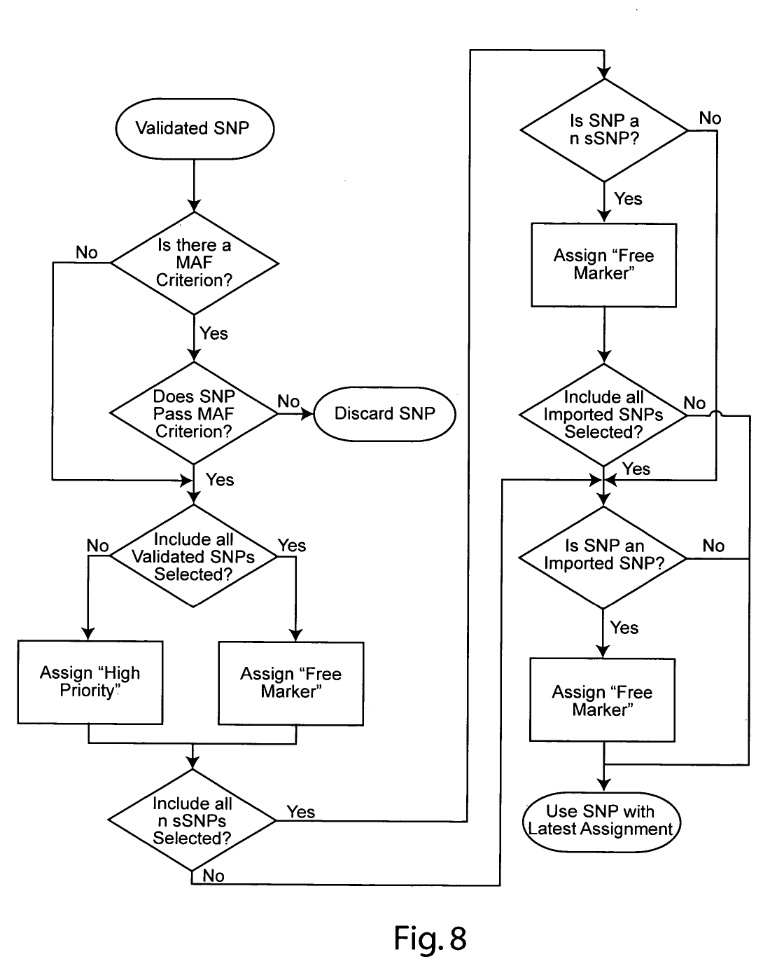

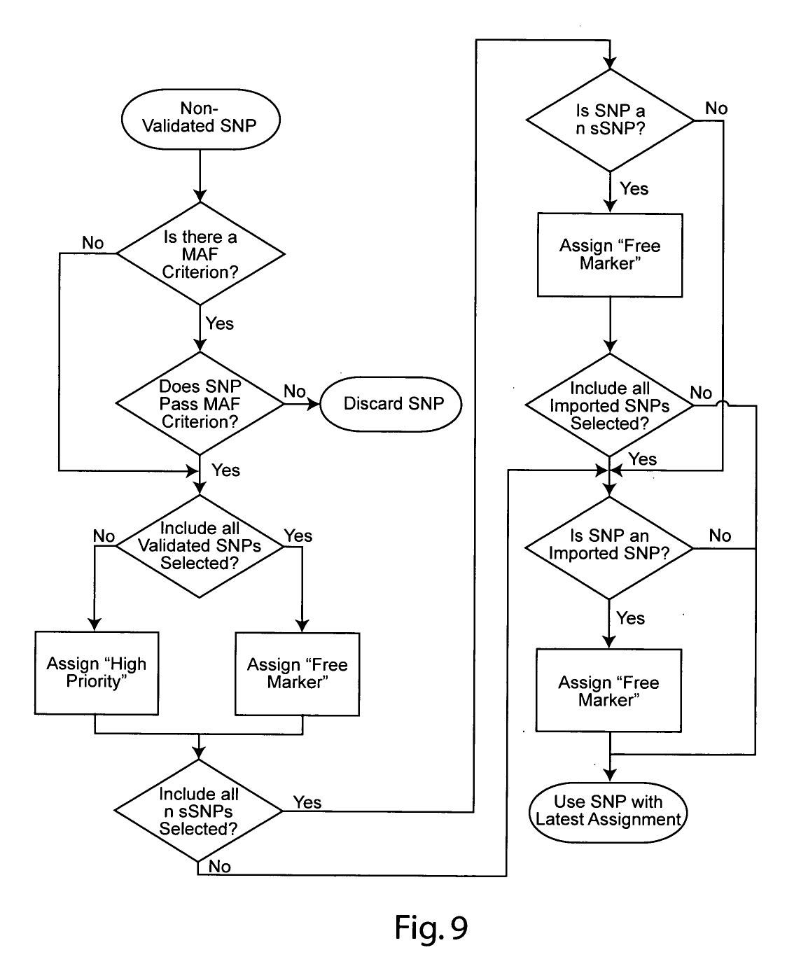

Priority type is assigned to each SNP differently depending if the SNP is validated (and has an off-the-shelf assay available; see FIG. 8), or non-validated, i.e. is a putative SNP discovered in silico and/or from small sample size and its conversion potential into a working assay is yet to be determined (see FIG. 9).

Exemplary MAF Criteria Definition

When the minor allele frequencies (MAF) determined in reference populations are used in the prioritization, MAF from 2 or more populations can be combined to define a MAF prioritization criterion. Boolean operators like “AND” and “OR” can be applied for this purpose, and the validated or non-validated status of the SNP can be used to bias this definition. Finally, missing data should be deal with appropriately (i.e. not in all cases MAF information is available for the 2 or more populations. The following describes the algorithms used to define the MAF criteria in presence of Boolean operators, SNP validation status, and missing data.

For Validated SNPs:
If “and” is specified: The SNP's Minor Allele Freq. has to be greater of equal to the cutoff value for all selected populations. An “N/A” counts as a zero.
If “or” is specified: The SNP's Minor Allele Freq. has to be greater of equal to the cutoff value for at least one of the selected populations. An “N/A” counts as a zero.

For Non-Validated SNPS:
The “Strong MAF Criterion” is computed the same as for validated SNPs.
For the “Weak MAF Criterion”, however, an “N/A” will count as 50 (i.e. always pass), unless all four populations are “N/A” in which they will count as zero.

Step 5: Selection of the Fewest Number of SNPs to Meet the Required Spacing Taking into Account the Prioritization Scheme

In this step an algorithm to select a subset of the SNPs that meet the spacing target is executed. If, for example, the target density is 10 kb, SNPs will be added in an evenly spaced fashion until the largest gap is less than or equal to 10 kb. Gaps are defined as the distance between consecutive SNPs, as well as the distance from any of the edges of the current view to the closest SNP. The algorithm takes into account the prioritization schema defined in Step 4 trying to maximize the selection of the highest priority SNPs over low priority when picking markers. This may be considered a modification of a “markerSpacing” algorithm. The modification allows the algorithm to take into account the prioritization scheme of Step 4.

When multiple SNPs occupy the same location (e.g. in the LD map coordinates is common to find segments of zero LDU), a preprocessing algorithm is applied before the markerSpacing algorithm as follows (Note: The SNPs are always kept in a sorted order of increasing position.):
1. Find a SNP with the same position as the SNP immediately following it, and with different priority types assigned.
2. Remove (filter out) the SNP with the lower priority
3. Repeat steps 1 and 2 until no SNPs are found which satisfy step 1's criterion.
4. Find a group of consecutively indexed SNPs with the same position, and with a priority assignment which is not “free marker”. Note: due to the execution of steps 1, 2, and 3, this group is guaranteed to all have the same priority type assignment.
5. Keep just the one SNP in the median index position, and remove all the other SNPs in this group.
6. Repeat steps 4 and 5 until no SNPs are found which satisfy step 4's criterion.

Only SNPs that survived the pre-processing algorithm are submitted to markerSpacing for final density selection.

Step 6: Visualizing the Result of the Marker Selection

Once the algorithm has picked markers, a visualization device indicates the selected markers over the background of all candidate SNPs. Typically, a different color is used to highlight the selected markers on a visualization panel showing the coordinate system, location of SNPs, and other features like genes and their exons (FIG. 8). Visualization panels can be offered summarizing the number and composition of markers selected (e.g. Validated vs. non-validated), and highlighting whether the largest spacing gap meets the requirements and the location of this gap.

Step 7: Fine Tuning of Some of the Selection Parameters Based on Visual Feedback and Reselection of Markers

Based on the visual inspection of the results of the selection, a user may want to fine tune or change some of the selection parameters. This can be accomplished either starting again at the beginning of the workflow, stepping back on the decision chain to the step where the modification is sought, or through a device that allows the user to modify interactively some of the major criteria (e.g. spacing and MAF cut-off; see FIG. 9). During interactive modification the user can observe the effect of the changes on the selected markers through the visualization devices outlined on Step 6.

Step 8: Create Final List of Selected SNP Markers

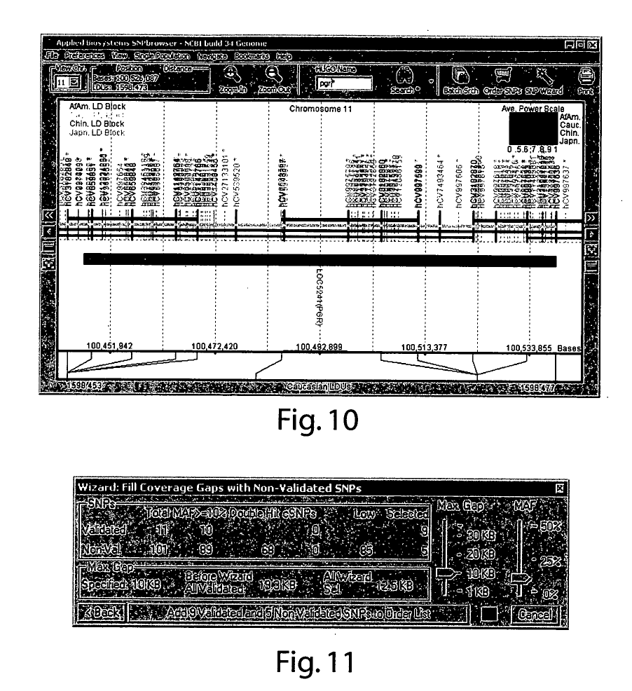

Once the user is satisfied on the selection of markers, these can be added to a list of SNPs for the study, and/or to a “shopping basket” for subsequent ordering of assays (FIG. 10). The list can be saved, or explored though a visualization device allowing panning and zooming to the genomic location of the markers in the list.

Step 9: Order Assays for Selected Markers, e.g. Linking to Online Store

With the list of SNP markers finalized, the user can order assays for these SNPs in a variety of ways: Placing the order over the phone, linking into an online store, e-mail, or cutting-and-pasting over an electronic order form.

II. SNP Tag Selection Workflow

In one embodiment, the SNPbrowser software includes a tool we call the Tagging Wizard. The Tagging Wizard allows the selection of a minimum informative subset of Validated SNPs, by removing SNPs providing redundant information due to strong LD with other markers. The resultant set of SNP “tags”, when genotyped in a study, should provide information on the non-genotyped SNPs with some level of information. The Wizard can reduce the numbers of Validated SNPs only, since genotype data in a reference panel is needed to assess the LD relationships between markers. Tag SNPs are inherently population-specific, although overlap of tags between populations may exist.

This workflow may be comprised of the following steps:
1. Select genomic region(s) of interest
2. Select SNP correlation metric to use as selection criteria
3. Select secondary criteria to filter candidate SNPs, e.g. minor allele frequency threshold
4. Select degree of correlation between SNPs
5. Selection of the fewest number of SNPs that meet the required correlation criteria
6. Visualizing the result of the marker selection
7. Fine tuning of some of the selection parameters based on visual feedback and re-selection of markers
8. Create final list of selected SNP markers
9. Order assays for selected markers

Steps 3 and 6 to 9 are optional. A more detailed description of each step, alongside snapshots of a “wizard”-like software implementation follows.

Step 1: Selection of Genomic Region(s) of Interest.

The first step involves selecting the genomic region of interest. Typically this would be a contiguous chromosomal segment including one or more genes, but it could encompass an entire chromosome or genome. These regions are usually derived from a list of candidate genes for an association study, or can also be derived from candidate regions resulting from a previous linkage mapping study (see FIGS. 3-5).

Step 2: Select SNP Correlation Metric to Use as Selection Criteria

Next, a correlation metric is selected to assess the statistical correlation of close by markers that would be used by the tag SNP selection algorithm (see FIG. 13). Correlation metrics try to quantify the degree of linkage disequilibrium between markers as well as the information that one marker, or a combination of markers, carry about a given SNP.

Correlation is usually calculated only if the set of candidate SNPs have been previously genotyped on a panel of DNAs from a representative sample of subjects from the population of interest. The correlation metrics currently in use to select tagging SNPs can be classified as follows:
Metrics that require phased haplotypes as input, and
Metrics that require raw genotypes as input

In the case of the metrics that require phased haplotypes, since is difficult to directly obtain haplotype information experimentally, typically a haplotype inference algorithms is used to deduce haplotypes from genotype data.

Also, metrics can also be classified as follows:
Pair-wise metrics, if they only consider pairs of SNPs at a time, and
Multivariate metrics, if they can consider multiple SNPs at a time

The following is a non-complete list of metrics that are currently in use in the field. Some or all of these may be implemented in the SNPbrowser wizard.

(a) Genotype Correlation. This metric allows the removal SNPs based on the correlation of genotypes between the SNPs in the view as obtained on a sample of the selected population. This is a pair-wise metric that requires genotypes as input. More details of this new algorithm are presented in the next section below.
(b) Pairwise r2. This is a classical measure of LD used in population genetics. Allows selection tag SNPs that maintain a minimum pair-wise r2 value with at least one removed SNP (See, e.g., Carlson et al, “Selecting a maximally informative set of single-nucleotide polymorphisms for association analysis using linkage disequilibrium. Am. J. Hum. Genet. 74:106-120 (2004). This is a pair-wise metric that requires genotypes as input.
(c) Haplotype Informativeness. Metric that evaluates an informativeness value of the haplotypes inferred on a sample of the selected population (See, e.g., Halldorsson et al, “Optimal haplotype block-free selection of tagging SNPs for genome-wide association studies,” Genome Res In Press (2004). This is a multivariate metric that typically requires phased haplotypes as input, but can be extended to genotypes.

(d) Haplotype R². This option assesses the haplotype R² value of the haplotypes inferred on a sample of the selected population (See, e.g., Weale et al., “Selection and evaluation of tagging SNPs in the neuronal-sodium-channel gene SCN1A: implications for linkage-disequilibrium gene mapping,” Am J. Hum Genet 73:551-565 (2003). This is a multivariate metric that requires haplotypes as input.
(e) Haplotype Entropy. This metric allows to asses the information content that a SNPs contributes relative to the haplotype diversity of the (common) haplotypes of the region measured as entropy. Typically applied to LD/haplotype blocks is a multivariate metric that requires phased haplotypes as input. In a previous disclosure (No. 4946) we presented an efficient algorithm to calculate this metric and use it on tag SNP selection (See, e.g., Avi-Itzhak et al, “Selection of minimum subsets of single nucleotide polymorphisms to capture haplotype block diversity,” Pacific Symposium on Biocomputing. World Scientific Press, Lihue, Hi., pp 466-477 (2003).
(f) Statistical power. Another possible metric to optimize during the selection of markers is the statistical power of finding an association given the type of test, sample size, and assumed architecture of the disease or trait. The assumptions made would include mode of inheritance, penetrance and prevalence, type of test (marker by marker or haplotype), number of causative mutations, MAF of causative mutations, sample size and type (case/control vs. trios or sib pairs), etc. This metric could be estimated from raw genotypes, or from haplotypes, and can be implemented as a pair-wise, or multivariate metric (See, e.g. Hu et al., “Selecting Tagging SNPs for Association Studies using power calculations from genotype data,” Human Heredity 57 (2004).

Other metrics and extension of the previous are feasible. See, e.g., Weale et al, cited above.

Exemplary Genotype Correlation Algorithm

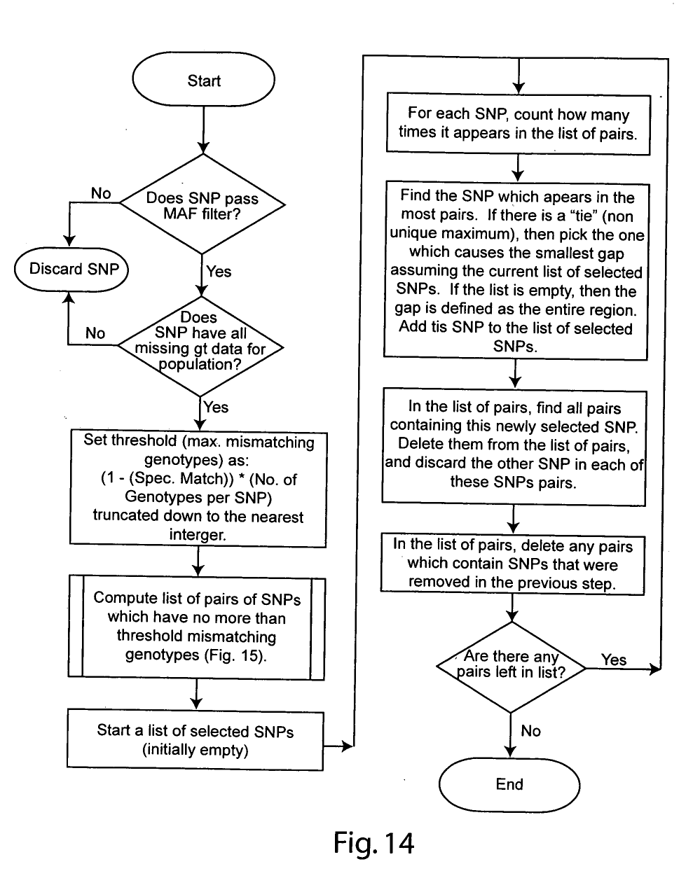

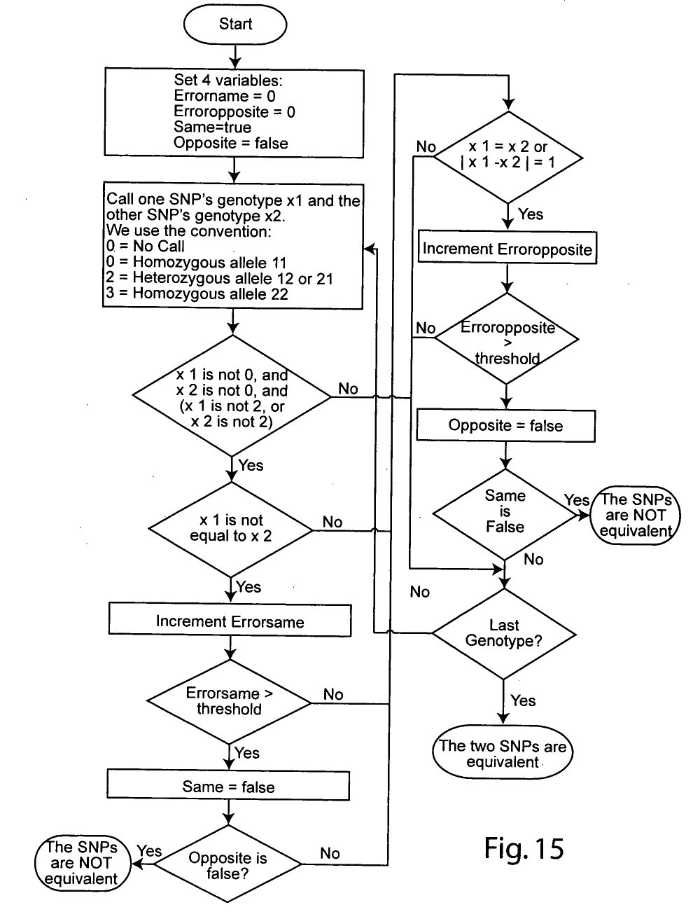

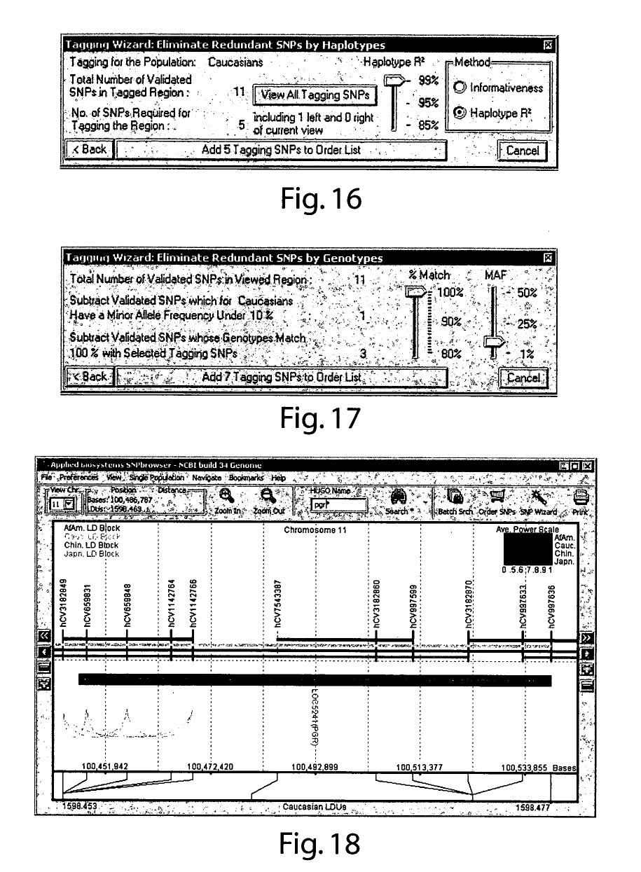

The selection of minimum informative subsets of SNPs based on genotype correlation is original to the present teachings. SNPs are removed by assessing their genotype correlation with other SNPs, leaving in the final list the SNP “tags”. This correlation is computed on a per population basis based on genotypes obtained on reference samples (the same samples for the SNPs being used). FIGS. 14 and 15 present the details of the algorithm implemented in SNPbrowser.

Some additional heuristics that we use in the current implementation include the following:
When comparing all pairs of SNPs, one doesn't have to look beyond a certain distance which can be either kb, LDUs, or number of SNPs, or the min of any of them. For very large regions this will increase speed a lot. For a typical region like a gene with less than 300 SNPs it will have no speed improvement. In SNPbrowser we use 300 SNPs as the maximum distance.
When comparing the genotypes of a pair of SNPs, there is no need to perform the calculation if their minor allele frequencies are to disparate (this improves speed a lot). In SNPbrowser we use the following empirically derived rules:
If a perfect match (threshold=0) is required, the if the minor allele frequencies of the two SNPs being compared are more than 16 percentage points apart, then we decide that the two SNPs are not equivalent without actually comparing genotypes.
For the other match settings (85 to 99 percent) a threshold of 22 may be used.

For optimum speed, specific threshold values can be derived for each percentage match.

Step 3: Select Secondary Criteria to Filter Candidate SNPS, e.g. Minor Allele Frequency Threshold

Optionally, a secondary criteria can be fixed at this or later stage of the workflow to exclude SNPs from the selection procedure. Typically, a threshold of MAF would be used to exclude less informative SNPs with frequencies lower than 10%. In addition, a statistical test to detect deviation from Hardy-Weinberg equilibrium can be applied to exclude SNPs were potential genotyping error has occurred.

Step 4: Select Degree of Correlation Between SNPs

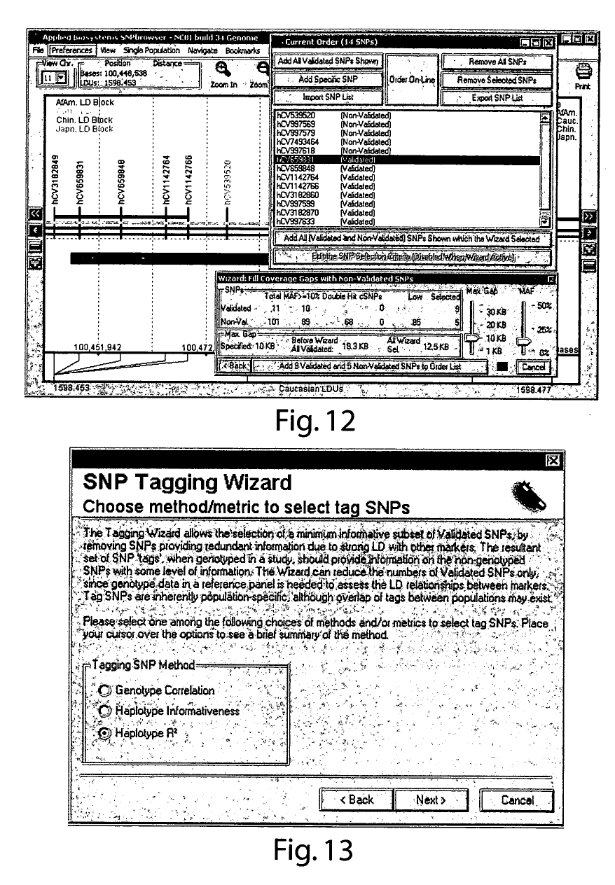

After selecting the correlation metric and starting set of SNPs, a degree of correlation is selected above which the selection algorithm will pick SNPs for genotyping in the study, that represent the unselected SNPs to a certain quality value. For the metrics described above, this typically ranges from 85-100% of the maximum value possible for each metric (FIGS. 12-13). At 100% correlation, the tag SNPs would, in most cases, faithfully predict the status of the unselected, or tagged SNPs. Less than that, some level of information is lost and the researcher may want to consider certain levels of loss in order to achieve a reasonable cost of the study without losing too much power. In one implementation of the wizard in SNPbrowser (see FIG. 12), we offer 99% as the maximum value for the haplotype-based methods, since this produces significant savings by only loosing information on the very rare haplotype.

Step 5: Selection of the Fewest Number of SNPs that Meet the Required Correlation Criteria

At this stage an algorithm to select the minimum informative subsets of SNPs that meets the correlation specifications is executed. This could be executed off-line, due to computational requirements, or real-time. If the algorithm is executed offline, from a pre-selected starting set of SNP and genotype data, the previous steps simply select from results previously executed and this step is reduced to locate the results. The latter is the current implementation of the SNPwizard for the haplotype-based methods, as haplotype inference from genotype data with statistical methods can be computationally intensive. In the case of the genotype correlation the selection algorithm is performed real-time from genotype data stored in the application.

The selection algorithm implementation can be “greedy’, which does not guarantee an optimal result but is fast, or optimal, involving exhaustive searches across the solution space, or through the use of dynamic programming. One algorithmic framework to select an optimal set through dynamic programming is described in Halldorsson et al, cited above. Such a framework may be used for the haplotype-based methods of a wizard implementation within a SNPbrowser.

Step 6: Visualizing the Result of the Marker Selection

Once the algorithm has picked markers, a visualization device indicates the selected markers over the background of all candidate SNPs. Typically, a different color is used to highlight the selected markers on a visualization panel showing the coordinate system, location of SNPs, and other features like genes and their exons. Visualization panels can be offered summarizing the number and composition of markers selected (e.g. Validated vs. non-validated). Furthermore, other visualization cues can be used to highlight the relationships between the SNP tags and the tagged SNPs (e.g. arcs from the tag to the tagged; see FIG. 14).

Step 7: Fine Tuning of Some of the Selection Parameters Based on Visual Feedback and Reselection of Markers

Based on the visual inspection of the results of the selection, a user may want to fine tune or change some of the selection parameters. This can be accomplished either starting again at the beginning of the workflow, stepping back on the decision chain to the step where the modification is sought, or through a device that allows the user to modify I interactively some of the major criteria (e.g. correlation criteria and threshold). During interactive modification the user can observe the effect of the changes on the selected markers through the visualization devices outlined on Step 6 (FIGS. 12-13).

Step 8: Create Final List of Selected SNP Markers

Once the user is satisfied on the selection of markers, these can be added to a list of SNPs for the study, and/or to a “shopping basket” for subsequent ordering of assays. The list can be saved, or explored though a visualization device allowing panning and zooming to the genomic location of the markers in the list (See FIG. 10).

Step 9: Order Assays for Selected Markers, e.g. Linking to Online Store

With the list of SNP markers finalized, the user can order assays for these SNPs in a variety of ways: Placing the order over the phone, linking into an online store, e-mail, or cutting-and-pasting over an electronic order form.

III. Combinations and Variations of the Basic Workflows

In some circumstances may be desirable to combine the two previous workflows sequentially in order to select tagging SNPs and additional SNPs to cover the gaps in coverage from the original starting SNP set where the tagging was performed. This is desirable when a fully comprehensive list of SNPs with genotypes on population panels is not available, as is the case today. For example, the TaqMan Assays-on-Demand (AoD) set of validated SNPs, available from Applied Biosystems, is a gene-centric map, and thus tagging SNPs may be selected on the gene regions, but if SNPs are desired across an entire candidate region, supplementary SNPs can be selected on the basis of density. Furthermore, due to the empirical profile of LD, the AoD set may not cover perfectly all regions (e.g. gaps with less than one SNP per LDU). These supplementary SNPs would be desirable even after tag SNP selection. In such scenarios the combination of the above workflows is straightforward, and the only provision is to ensure that for the density selection both tag and tagged SNPs are considered as preexisting markers (e.g. select include all validated assays on the wizard prioritization panel).

Other variants to the workflow that can be envisioned include:
Selection of two target SNP densities according the gene content. For example, on a candidate region derived from linkage, one may want to select a high density of markers across and around the annotated genes (e.g. 10 kb), but on the intergenic regions one may want to include some markers at a lower density (e.g. 25 kb), to account for our imperfect knowledge of the location of all functional elements on the genome.
Combination of density selection for segments where LD is not high (e.g. LDU >0.1), with the use of a haplotype tagging method for the blocks of LD (i.e. LDU <0.1). This could be considered “the best of both worlds.”
Use of scaling factors that convert between the LD maps of one population where a representative panel has been genotyped across the genome, to the unknown map of another new population. An example would be to transform the map of a Caucasian outbred population, to population isolates after performing a pilot study to measure the scaling factor, and the use that extrapolated map to select SNP markers.
Instead of selecting markers one by one on each region/gene of interest, one may envision a batch workflow starting from a list of genes (candidate gene list) which is executed after choosing all the criteria and parameters. At the end of the process a summary of the selected markers for each gene is presented with the option of submitting to the shopping basket all of them, or to jump to each region for fine tuning and verification.
Biasing the selection of SNPS to markers within certain MAF interval. For example, everything else being equal, markers can be selected based on allele frequency when using the density selection but a number of SNPs are located within zero LDUs. In another example, an additional bias can be introduced in the SNP prioritization such as markers within an interval of MAF have higher priority.
Similarly to the previous variant, when selecting by density, markers can be selected to maximize the power for finding an association when certain mode of inheritance and architecture of the disease or trait is assumed.

IV. SNPbrowser Software—Simplifying tSNP Selection

In one embodiment, the SNPbrowser provides a graphic view of more than five million SNPs and includes genotype data generated from the Applied Biosystems database of 160,000 validated SNPs. In this embodiment, the SNPbrowser also includes genotype data generated as part of the HapMap Project. The tool includes pre-calculated LD maps, LD blocks, and tSNP sets. It also allows researches to download the genotypes that were used to calculate these elements. With easy access to these genotypes, researches can also, if they wish, calculate LD and tSNP sets within the tool, or visualize LD patterns using their own algorithms.

LD Blocks

LD blocks describe regions of extensive LD and low haplotype diversity. Many methods have been described to identify blocks. These haplotype blocks provide a conceptually simple model for understanding tSNPs and how a reduced set of SNPs can still report most haplotypic information. Two factors must be considered if haplotype blocks are used for tSNP selection:
LD block definitions depend on the algorithm used, and their boundaries are arbitrary and sometimes fuzzy.
LD blocks are useful for selecting tSNPs only for SNPs contained within them, and not for SNPs located between haplotypic blocks.

As an alternative to these ad hoc haplotypic block definitions, the genotypic data set may be used to calculate linkage disequilibrium units (LDUs), which define a metric coordinate system in which locations are additive and distances are proportional to the allelic association between markers. One LDU represents the LD decay between two SNPs by approximately 37% of its local maximum value on the Malecot model. The physical distance corresponding to one LDU in a particular genomic region is known as the swept radius. It has been suggested that the swept radius is the maximum practical distance across which LD can be detected.

Thus, at least one SNP is needed per LDU. Usually, 2-3 SNPs should be selected per LDU to compensate for lack of SNP informativeness and for assay and experimental difficulties. The metric LDU map does not required LD blocks. In a region with less recombination (i.e., more blocks), fewer LDU will be found than in a region of high recombination (i.e., fewer blocks). SNPbrowser Software LD blocks are defined either by LDU (one block equals all SNPs within 0.3 LDU or less), or by an alternative, rule-based method previously described in the literature.

Using SNPbrowser Software

In one embodiment, an SNPbrowser tool, constructed in accordance with the teachings herein may be used to visualize more than five million SNPs, including 160,000 SNPs validated by Applied Biosystems, which are available as off-the-shelf, validated TaqMan® SNP Genotyping Assays, as well as the SNPs genotyped by the International HapMap Project and additional SNPs. The software can be used to select markers for the SNPlex™ Genotyping System, because the population validation data from these SNPs is still applicable. SNPbrowser Software contains an additional 2.5 million SNPs that have passed all in silico design and genomic specificity rules for conversion to functional TaqMan assays. These SNPs can also be submitted to the SNPlex System assay design pipeline to obtain multiplex assays. Among the chief features and benefits of SNPbrowser Software are the following:
Data are displayed in the context of physical and LD maps, LD blocks, genes, and chromosomes.

The software contains SNP wizards—three easy-to-use tools for SNP selection:
1. Genotype correlation wizard: Removes SNPs with exactly correlated genotypes.
2. Density selection wizard: Traditional picket-fence distribution based on kilobase or LDU maps.
3. tSNP selection wizard: Selects SNPs by pairwise r² and haplotype R² methods.
   SNPbrowser Software allows genotypes to be exported for each validated SNP visible in the window. Data for all four populations are downloadable. The Caucasian and African-American DNA samples analyzed by Applied Biosystems can be obtained from the Coriell cell repositories. This allows researches to use the data either as a control for comparing results or as an addition to their own data, generated from the same samples and used for their own calculations.

Genotype Correlation

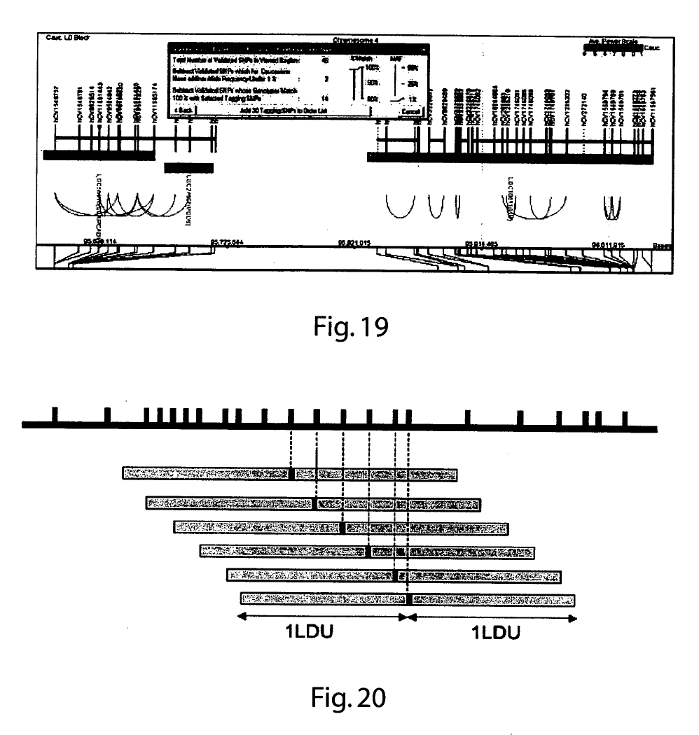

The genotype correlation wizard in SNPbrowser Software allows researchers to select the simplest possible tagging set by simply removing SNPs that correlate 100% to other SNPs (i.e. r²=1). If the wizard is set at the recommended setting of 100% identity, SNPs that have identical genotypes in the selected population sample will be removed (FIG. 19 and Table 1).

TABLE 1
Genotype Correlation Wizard

| Sample | SNP 1 | SNP 2 | SNP 3 | SNP 4 |
|---|---|---|---|---|
| 1 | 1.1 | 1.1 | 2.2 | 1.1 |
| 2 | 2.2 | 2.2 | 2.2 | 1.1 |
| 3 | 2.2 | 2.2 | 1.1 | 2.2 |
| 4 | 1.2 | 1.2 | 1.1 | 2.2 |
| 5 | 2.2 | 2.2 | 2.2 | 1.1 |

In the above table, the results for SNP 1 and SNP 2 are identical; therefore, only one needs to be typed. The results fro SNPs 3 and 4 are reversed, but if the results are known for one of them, the results can be predicted for the second one. By typing SNP 1 and SNP 3, no information is lost, as SNP 2 and SNP 4 are 100% correlated with these SNPs, respectively. The correlation threshold can be reduced below the default 100% correlation value, and in this way, the tSNP set can be reduced; however, it should be noted that the loss in power incurred below this level is not well understood, and, thus, it is not recommended.

The Pairwise r2 Method

To understand how the SNP wizard is able to suggest a set of tSNPs, it is necessary to review the tSNP selection process. The pairwise r² method requires the following three steps:
1. Determine meaningful LD regions or windows to allow tSNP selection to be performed on SNP sets that can be expected to inform each other.
2. Select tSNPs that are correlated in a pairwise fashion with another SNP in the window; then, using r² as the quality metric, determine the quality of each tSNP set by assessing how well the tSNP and the tagged SNP correlate.
3. Optimize the number of tSNPs in the final set by selecting from all possible alternative tSNP combinations in each window; the combination that results in the smallest possible number of tSNPs for each chromosome.

Determining LD Regions

The pairwise r² method determines tSNPs for each SNP on the complete starting set. This step does not required the strict definition of haplotype blocks, although it is clear that within regions of high LD, the selection of a smaller tSNP set is more effective. It is not necessary to assess tSNPs if two SNPs lack allelic association because of ancestry. Each SNP is assessed in a sliding window of SNPs (FIG. 20). Only SNPs with minor allele frequency (MAF) values >5%, and which have passed the Hardy-Weinberg equilibrium test with a p value >0.05 are considered for tSNP selection.

To calculate tSNP sets, it is necessary to use a series of sliding windows for the region that is being tagged. This is necessary because:
It is not useful to include SNPs >1 LDU from the SNP being tagged.
The computational problem is a non-deterministic polynomial (NP) hard problem that is not solvable in a reasonable time frame; thus, the number of SNPs must be restricted to within 1-LDU window. Additionally, the physical distance cannot exceed 200 kb, and the number of SNPs is limited to 12 per window.

For association mapping applications, only SNPs that reflect regions of common ancestry are of interest, rather than distinct SNPs that may be in LD from admixture, selection, or chance. The regional selection method (200,000≦6 SNPs 1 LDU) ensures that only reasonably close SNPs are chosen, thereby restricting SNP analysis to those for which an observed allelic association results from common ancestry.

Calculating the Pairwise r2 Value

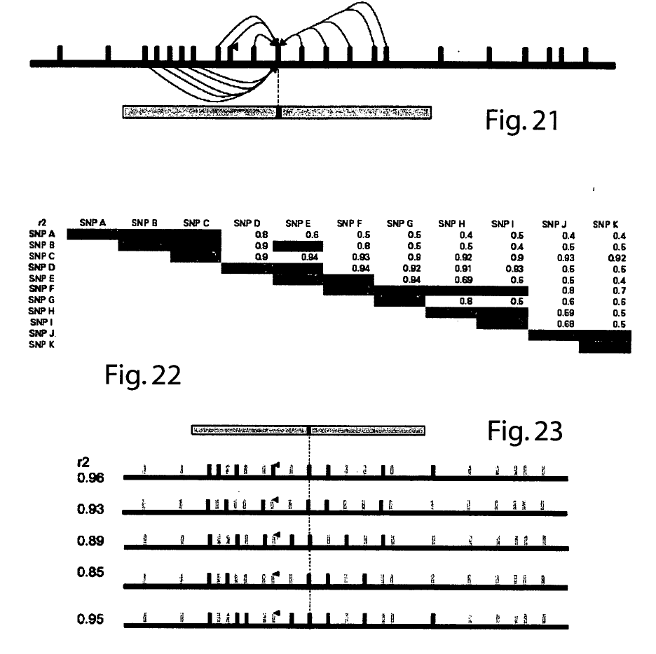

Because each SNP can be tagged by any other SNP in the window, genotypes are used to calculate the pairwise r² value between the target SNP and each SNP in the window (FIG. 21). An SNP can be considered to tag another SNP only if the r² value passes a user-defined threshold. Each region may contain multiple alternative combinations of tSNP sets, which must also be assessed. Note that if an SNP cannot be tagged by any other SNP, it is included in the final set of tSNPs that will be typed. Also, two or more SNPs in the window can sometimes tag a given target SNP equally well (i.e., above the specified threshold). In this case, all possible alternatives will be saved for the optimization, which will be performed in the subsequent step at the chromosome level.

Selecting a Minimal SNP Set

After evaluating all possible tSNPs for the entire chromosome, several alternatives are possible, and they must be evaluated to select a minimal optimal subset of tSNPs. For example, if an SNP can tag more than two independent SNPs, it is a preferred tSNP compared with two tSNPs, each of which tags the two target SNPs. Thus, one can select a minimal subset of SNPs that tag the entire haplotype with an r² value greater than, or equal to, the required threshold. Obviously, if an SNP cannot be tagged by any other SNP, it should be included in the final set (i.e., it tags itself).

FIG. 22 shows the selection of a minimal set. Because all tSNPs data have been pre-calculated in SNPbrowser Software, it is possible to select the optimal SNP set for the whole chromosome. When a researcher selects a region in SNPbrowser Software, tagging SNPs are provided that detect all SNPs in the window.

Haplotype R2 Method

The haplotype R² and pairwise r² methods are identical, except that the haplotype R² method is based on a multivariate metric that calculates the correlation between multiple SNPs. The pairwise r² method, a more conservative approach, does not calculate possible simultaneous correlations between multiple SNPs and thus it usually selects more SNPs than the haplotype R² method.

These calculations require phased haplotypic date. To acquire it, haplotypes are inferred for each window, using a maximal likelihood/expectation method that accurately infers all major haplotypes from the available genotypic data. This method relies on the common disease/variant hypothesis in which common haplotypes will be associated with phenotypes. If the phenotype of interest is caused by many rare alleles, they will be found on rare and possibly undetectable haplotypes. For each SNP, there will now be a set of tagging SNPs (FIG. 23).

Gap Filling—Another Task Facilitated by SNPbrowser Software

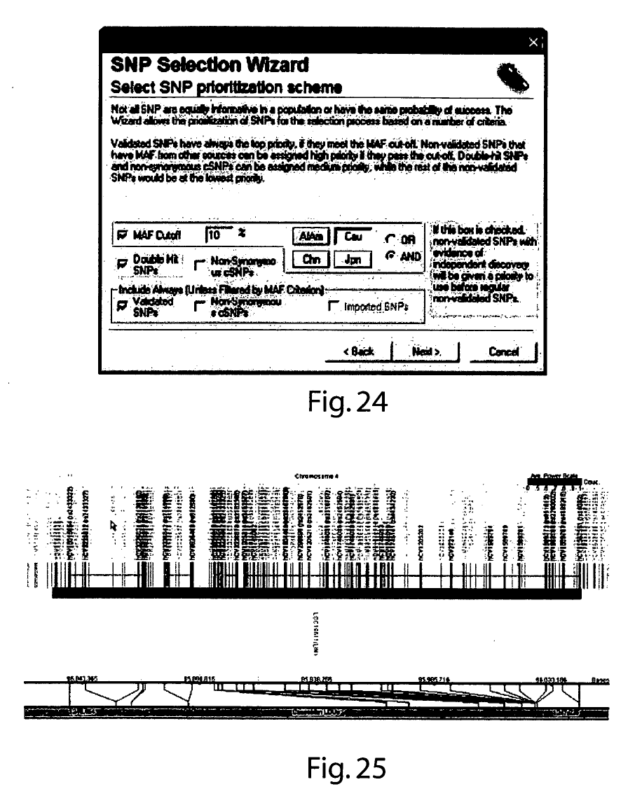

If a region contains no genotyped SNPs within 1 LDU of each other, selecting a set of tSNPs to cover this region will be impossible and that region will remain untagged. For these gap regions, SNPbrowser Software offers a density-selection wizard, which allows the selection of an equally spaced (picket-fence) SNP set (FIG. 24).

Spacing can be determined by kilobase or by LDU (the recommended method). Because selection is based on the distance between SNPs, which does not require genotypes, all five million SNPs in SNPbrowser Software can be used. These SNPs consist of the 160,000 SNPs validated by Applied Biosystems and the HapMap project, as well as an additional two million SNPs that have human SNP assays that pass all the in silico design and genome specificity rules, providing researchers with an unprecedented selection of markers across the genome. Furthermore, SNPs used in the selection process can be prioritized.

It should be noted that in the density selection method, based on LDU coordinates for untyped SNPs, map positions are linearly interpolated from the values of adjacent typed markers. This may introduce some error, but this selection method is still preferable to using physical distance, which has little correlation with LD patterns. In addition, as additional data from the HapMap project becomes incorporated into SNPbrowser Software, the number of untyped markers and the error of interpolation will substantially reduce.

Using SNPbrowser Software for tSNP Selection—an Example

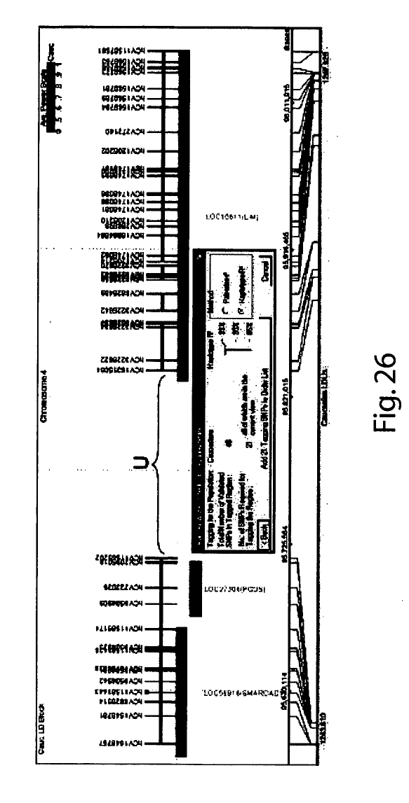

The process for selecting the minimal number of SNPs for an association study is described in FIGS. 25-25, and the results can be found in Table 2. The study involves the LIM gene from the Caucasian population sample region in FIG. 25 (chromosome 4; 95,582,389-96-96,059,640 bp). The work-flow demonstrates how SNPbrowser Software and the tSNP wizard (FIG. 26) combine the density selection process with tSNP selection to determine the best method. Table 2 shows the number of SNPs required to tag the LIM gene, as defined by the haplotype R² and pairwise r² methods, at a variety of thresholds (85%, 95% and 99%).

TABLE 2
Results for Six Possible Choices

| Haplotype R² | | | | Pairwise r² | | |
|---|---|---|---|---|---|---|
| | Total | Genotypes/ | | Total | Genotypes/ | |
| | SNPs | 1,000 Samples* | | SNPs | 1,000 samples | |
| No selection | 30** | 30,000 | 100% | 30** | 30,000 | 100% |
| 0.99 r² | 14 | 14,100 | 47% | 23 | 23,230 | 77% |
| 0.95 r² | 13 | 13,650 | 45% | 19 | 19,950 | 67% |
| 0.85 r² | 9 | 10,620 | 35% | 13 | 14,340 | 48% |

**Although 32 SNPs are present, two have no measured minor allele frequency in Caucasians; therefore, they are not considered in the tSNP calculation.
*The number of genotypes is calculated by multiplying the number SNPs (i.e., the number of samples ¥he increase in sample size).

A Software Implementation Block Diagram

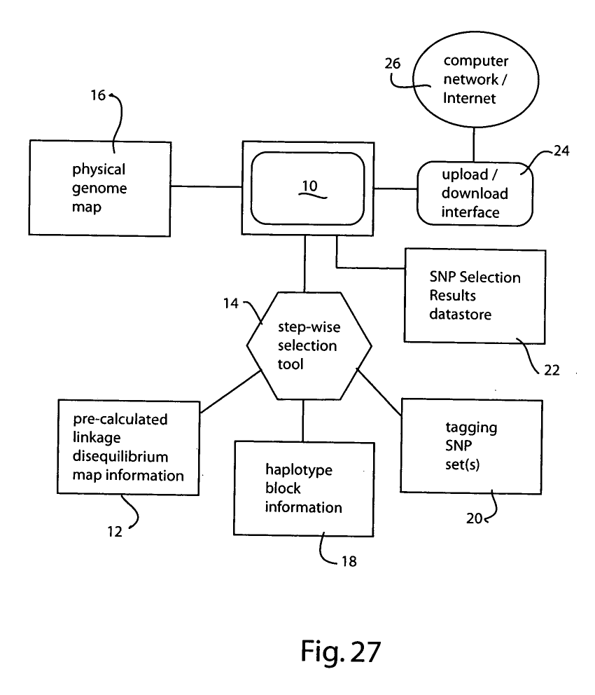

From the foregoing, it will be appreciated that a visual software tool can provide a number of significant advantages in the selection of SNPs for genotyping experiments. For a further understanding, refer now to FIG. 27, which illustrates some of the components that may be used to implement such a visual tool.

As illustrated, the tool preferably includes a graphical user interface 10 on which a visualization of SNPs may be integrated with a physical genome map. Data to display such a physical genome map may be stored in the physical genome map datastore 12. The step-wise selection tool 14 communicates with the interface 10, and allows the user to selectively employ the various techniques discussed in detail above, to select SNPs, make SNP tag selections and control SNP density. The step-wise selection tool obtains data from the pre-calculated linkage disequilibrium map information datastore 16, the haplotype block information datastore 18, and the datastore 20 containing at least one set of tagging SNPs.

As the user works with the step-wise selection tool, to define the desired set of SNPs for his or her experiment, the results are stored in a results datastore 22, the contents of which may be displayed graphically on interface 10.

In one embodiment, an upload/download interface 24 couples the software tool to a computer network, such as a local area network, wide area network and/or the internet 26. Through this interface the user can send and receive information used by the tool to assist in SNP manipulation.

In addition, the tool may include a processing engine 28 that can be used for a variety of purposes. These include: (a) calculating linkage disequilibrium map information apart from the pre-calculated linkage disequilibrium map information stored in datastore 12; (b) permitting a user to define new sets of tagging SNPs; and (c) permitting a user to change the algorithms by which linkage disequilibrium map information is generated.

From the foregoing, it will be appreciated that, given the complexity and controversy in the criteria for selection of markers for genetic studies, a set of streamlined workflows implemented as a software wizards where options are selectable, would be an enormous help for researchers making decisions to set up their study. Rather than finding its way through the maze of data and applications in order to come out with a list of markers, the researcher would have access to all the necessary information on a single integrated interface. Currently, there are very few applications to help researchers in this task and they are restricted to a single specific aspect and do not provide the integration presented here.

The iterative nature of the process as presented in this teachings, together with the visualization feedback and cues proposed, is key to allow researchers to understand the consequences of the settings they select, as well as refine their criteria for selecting markers very quickly. This would accelerate the study set-up phase reducing the time to results. Furthermore, the understanding of the selection criteria gained through the workflows, would increase the probability of designing properly powered studies with greater probability of success.

Finally, since the process bias the selection to previously validated markers (e.g. SNPs) for which more information and sometimes a validated assay are available, these teachings would ensure a higher assay conversion and pass rates. Simultaneously, this would result in lower support costs for assays products (as less failures would be expected) and a preferential movement of AB off-the-shelf assay inventory over custom assays.

While these teachings have described a software tool that may be embedded as a wizard in a graphical browser, such as the SNPbrowser software for the selection of genomic assays, other embodiments are also possible. For example, these teachings may be readily extended to accommodate and/or include products such as Applied Biosystems TaqMan assays and the SNPLex SNP genotyping system.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

We claim:

1. A visual tool to facilitate selecting SNPs for genotyping experiments, comprising:
a first memory containing a datastore of pre-calculated linkage disequilibrium map information;
a second memory containing a datastore of haplotype block information;
a third memory containing at least one set of tagging SNPs;
a graphical user interface that provides visualization of SNPs integrated with a physical genome map;
a stepwise selection tool associated with said graphic user interface to facilitate selection of tagging SNPs by selectively using the information in at least one of said first, second and third memories.

2. The tool of claim 1 wherein said stepwise selection tool is adapted to selectively overlay onto said physical genome map, one or more of the following: (a) said pre-calculated linkage disequilibrium map information, (b) said haplotype block information, and (c) said set of tagging SNPs onto said physical genome map.

3. The visual tool of claim 1 wherein said stepwise selection tool is further adapted to select SNPs based on a predetermined spacing with respect to at least a portion of said physical genome map.

4. The visual tool of claim 1 further comprising an interface adapted to couple to a network and allow downloading of information relating to the genotypes used to develop at least one of said pre-calculated linkage disequilibrium map information, said haplotype block information and said at least one set of tagging SNPs.

5. The visual tool of claim 1 further including processing engine adapted to calculate linkage disequilibrium map information apart from said pre-calculated linkage disequilibrium map information.

6. The visual tool of claim 1 including processing engine adapted to permit a user to define new sets of tagging SNPS.

7. The visual tool of claim 1 including processing engine adapted to permit a user to change the algorithms by which linkage disequilibrium map information is generated.

8. The visual tool of claim 1 wherein said tool includes a genotype coorelation wizard adapted to remove SNPs based on genotype correlation.

9. The visual tool of claim 1 wherein said tool includes a density selection wizard adapted to define a uniformly spaced distribution of SNPs.

10. The visual tool of claim 1 wherein said tool includes an SNP selection wizard that selects SNPs using a pairwise r² method.

11. The visual tool of claim 1 wherein said tool includes an SNP selection wizard that selects SNPs using a haplotype R² method.

12. A method for determining SNP density for genotyping experiments, comprising the steps of:
selecting a genomic region of interest using a graphical visualization tool;
selecting a coordinate system within said tool;
selecting a desired target spacing;
using said tool to select a prioritization scheme of available candidate SNPs on said selected genomic region;
using said tool to select a minimized number of SNPs to meet said desired target spacing while taking into account said prioritization scheme; and
creating a final list of selected SNP markers and storing said list in a memory using said tool.

13. The method of claim 12 further comprising, using said tool to visualize the results of said step of selecting a minimized number of SNPs and using said tool to re-select at least some of said SNPs based on visual feedback.

14. The method of claim 12 further comprising, using said tool to visualize the results of said step of selecting a minimized number of SNPs and using said tool to fine tune at least some of the selection parameters based on visual feedback and then using the fine tuned parameters in re-selecting at least some of said SNPs.

15. The method of claim 12 further comprising, using said stored list of selected SNP markers to access an online store to order assays corresponding to at least one of said selected SNP markers.

16. The method of claim 12 wherein said step of selecting a genomic region of interest is performed by defining a contiguous chromosomal segment including one or more genes.

17. The method of claim 12 wherein said step of selecting a coordinate system is performed by placing markers on a physical genome map based on data accessed by said tool.

18. The method of claim 12 wherein said step of selecting a coordinate system is performed by placing markers on a linkage disequilibrium map based on data accessed by said tool.

19. The method of claim 12 wherein said prioritization step is performed by giving priority to validated SNPs.

20. The method of claim 12 wherein said prioritization step is performed so as to meet a minor allele frequency cut-off in a population of interest.

21. The method of claim 12 wherein said prioritization step is performed by giving priority to validated SNPs.

22. The method of claim 12 wherein said prioritization step is performed by assigning each SNP a prioritization type selected from the group consisting of: free marker, high priority, medium priority, low priority, no priority, and discard.

23. The method of claim 12 wherein said step of selecting a minimized number of SNPs to meet said desired target spacing is performed by measuring the gap spacing between SNPs, identifying the pair of SPNs having the largest gap and then adding SNPS in an evenly spaced fashion until the largest gap is less than or equal to a predetermined threshold value.

24. The method of claim 14 wherein the step of fine tuning at least some of the selection parameters is performed by adjusting the spacing or MAF cut-off parameters.

25. A method for performing SNP tag selection for genotyping experiments, comprising the steps of:
selecting a genomic region of interest using a graphical visualization tool;
using said tool to select an SNP correlation metric to use as a selection criteria;
using said tool to indicate a required correlation criteria by selects a degree of correlation between SNPs;
using said tool to select a minimized number of SNPs that meet said required correlation.

26. The method of claim 25 further comprising using said tool to apply a secondary criteria to filter candidate SNPs.

27. The method of claim 26 wherein said secondary criteria is based on a minor allele frequency threshold.

28. The method of claim 25 further comprising using said tool to visualize the results of said SNP selection and re-selecting at least some of said SNPs based on visual feedback.

28. The method of claim 25 further comprising using said tool to visualize the results of said SNP selection and fine tuning at least some of the selection parameters based on visual feedback.

29. The method of claim 25 further comprising creating a final list of selected SNP markers and using said tool to store said final list in a memory.

30. The method of claim 29 further comprising using said stored list of selected SNP markers to access an online store to order assays corresponding to at least one of said selected SNP markers.

31. The method of claim 25 wherein said step of selecting a genomic region of interest is performed by defining a contiguous chromosomal segment including one or more genes.

32. The method of claim 25 wherein said step of selecting an SNP correlation metric is performed by quantifying the degree of linkage disequilibrium between SNPs.

33. The method of claim 25 wherein said step of selecting an SNP correlation metric is performed using phased haplotype information.

34. The method of claim 25 wherein said step of selecting an SNP correlation metric is performed using raw genotype information.

35. The method of claim 25 wherein said step of selecting an SNP correlation metric is performed using a pair-wise metric that considers pairs of SNPs at a time.

36. The method of claim 25 wherein said step of selecting an SNP correlation metric is performed using a multivariate metric that considers multipe SNPs at a time.
